(19) United States
(12) Patent Application Publication
(10) Pub. No.: US 2008/0154357 A1
(43) Pub. Date: Jun. 26, 2008

(54) Multiport Vascular Prosthesis

(75) Inventor: Alon Shalev, RaAnana (IL)

Correspondence Address: Martin D. Moynihan;PRTSI, P.O. Box 16446, Arlington, VA, 22215, US

(73) Assignee: Nicast Ltd., Lod (IL)

(21) Appl. No.: 11/795,925

(22) PCT Filed: Jan. 25, 2006
(86) PCT No.: PCT/IL2006/000101

§ 371 Date: Jul. 24, 2007

Related U.S. Application Data
(60) Provisional application No. 60/646,537, filed on Jan. 25, 2005.

(51) Int. Cl. A61F 2/82; B29C 67/00

(52) U.S. Cl. 623/1.43; 623/1.54; 623/1.42; 264/451

(57) ABSTRACT

A vascular prosthesis, comprising a primary tubular structure of non-woven polymer fibers and a secondary tubular structure of non-woven polymer fibers, the primary and the secondary tubular structures being in fluid communication via an anastomosis such that the primary tubular structure terminates at the anastomosis and the secondary tubular structure continues at the anastomosis.

### FIELD AND BACKGROUND OF THE INVENTION

The present invention relates to implantable devices, and, more particularly, to a multiport vascular prosthesis.

Renal disease is an important cause of mortality and morbidity throughout the world. Renal disease may be acute or chronic. Acute renal failure is a worsening of renal function over hours to days, resulting in the retention of nitrogenous wastes (such as urea nitrogen) and creatinine in the blood. In comparison, chronic renal failure results from a loss of renal function over months to years.

Currently, hemodialysis is the primary modality of therapy for patients diagnosed as having end stage renal disease. Hemodialysis is the purification of blood by removing toxic substances and restoring chemical balance using an extracorporeal dialysis machine. The process is used as a substitute for proper kidney function. A hemodialysis machine pumps blood from the patient, through a cleansing solution and then back into the patient. Hemodialysis thus requires a constant flow of blood along one side of a semipermeable membrane with the cleansing solution on the other. Diffusion and convection allow the cleansing solution to remove unwanted substances from the blood while adding back needed components. In this manner, the cleansing solution removes the toxins and water from the blood by a membrane diffusion principle.

The dialysis machine is connected to a hemodialysis access site present in the patient's body. To be medically useful, the hemodialysis access site must remain unblocked and free from medical complications in order to enable dialysis to take place. The access site must also allow blood to flow to and return from the dialysis machine at a sufficiently high rate to permit efficient dialysis process. Known types of hemodialysis access sites include hemodialysis catheters and arteriovenous shunts.

A hemodialysis catheter is a percutaneous tube placed through the skin and directly into the vein which is typically the subclavian vein, internal jugular vein or femoral vein. The extracutaneous portion of the catheter is left in a certain position relative to the body ready and waiting to be used during an active dialysis session.

An arteriovenous shunt is a passage that redirects the flow of blood from an artery to a vein. Arteriovenous shunts can occur naturally in the body, but in connection to hemodialysis access sites they are typically refers to either an artificial connection between an artery and a vein. There are two types of arteriovenous shunts that are commonly practiced, an arteriovenous fistula and a prosthetic graft.

An arteriovenous fistula is a direct connection of an artery to a vein. The connection causes more blood to flow into the vein. As a result, most blood bypasses the high flow resistance of the downstream capillary bed, thereby producing a dramatic increase in the blood flow rate through the fistula. The vein is punctured repeatedly and the high blood flow permits hemodialysis to occur.

A prosthetic graft is an artificial vascular prosthesis surgically placed under the skin. Typically, prosthetic grafts are used in patients with small veins which cannot develop properly into a fistula. The graft is connected to an arterial source on one end and a venous source on the other end. The graft is accessed by the cannulas of the dialysis machine, to allow the blood to flow through the cannula into the dialysis machine, cleansed in the dialysis filter and then returned to the patient.

Despite the benefits of the hemodialysis, however, there are several drawbacks in conventional hemodialysis procedures. Access site complications, such as infection and access site failure, are believed to be the greatest cause of morbidity and mortality among renal disease patients. Many of access failures in arteriovenous fistulae and prosthetic grafts are caused by blood returning from the hemodialysis machine into the patient with sufficient high pressure to damage vein walls. Other complications are due to vein damage caused by traditional access methods resulting in risk for infection and clotting. In is recognized that the weak veins of renal failure patients may not accommodate certain access methods.

Artificial vascular prostheses, such as those enacting hemodialysis access sites, are well known and widely available in a variety of designs and configurations. Of particular interest are devices made of, or coated with, polymer materials which typically exhibit a microporous structure that in general allows healthy tissue growth and cell endothelization, thus contributing to the long term healing of the prostheses. Prostheses having sufficient porous structure tend to promote tissue ingrowth and cell endothelization along their inner surface.

Vascular prostheses presently used for hemodialysis access can be improved in two regards. First, it is desired that such prostheses will become surrounded by fibrotic tissue to reduce the risk of hemorrhage about the outer surface of the prosthetic graft following removal of the dialysis needle. Second, it is desired that such prostheses will have self-sealing properties, so as to minimize blood leakage following removal of the dialysis needle. A vascular prosthesis that offered an improvement of either of these regards without compromising other positive characteristics would be a significant step forward in the field of hemodialysis access.

A promising manufacturing technique of vascular prostheses is electrospinning. Electrospinning is a method for the manufacture of ultra-thin synthetic fibers which reduces the number of technological operations required in the manufacturing process and improves the product being manufactured in more than one way.

The process of electrospinning creates a fine stream or jet of liquid that upon proper evaporation of a solvent or liquid to solid transition state yields a nonwoven structure. The fine stream of liquid is produced by pulling a small amount of polymer solution through space by using electrical forces. More particularly, the electrospinning process involves the subjection of a liquefied substance, such as polymer, into an electric field, whereby the liquid is caused to produce fibers that are drawn by electric forces to an electrode, and are, in addition, subjected to a hardening procedure. In the case of liquid which is normally solid at room temperature, the hardening procedure may be mere cooling; however other procedures such as chemical hardening (polymerization) or evaporation of solvent may also be employed. The produced fibers are collected on a suitably located precipitation device and subsequently stripped from it. The sedimentation device is typically shaped in accordance with the desired geometry of the final product, which may be for example tubular, flat or even an arbitrarily shaped product.

The use of electrospinning for manufacturing or coating of vascular prostheses permits to obtain a wide range of fiber thickness (from tens of nanometers to tens of micrometers), achieves exceptional homogeneity, smoothness and desired porosity distribution along the coating thickness. When a graft is electrospinningly coated by a graft of a porous structure, the pores of the graft component are invaded by cellular tissues from the region of the artery surrounding the stent. Moreover, diversified polymers with various biochemical and physico-mechanical properties can be used in stent coating.

Although blood vessel implantation procedures are widely practiced and have become a routine procedure in hospitals throughout the world, they are not without certain operative limitations that would best be avoided. For example, in conventional prosthetic grafts used in hemodialysis, neointimal hyperplasia is caused when the cells of the inner layer of the vein hypertrophy and multiply in response to the high blood flow and pressure of the arteries. Neointimal hyperplasis results in the narrowing or “stenosis” of the distal outflow portion of the prosthetic graft device, and ultimately causes thrombosis of the entire length of the prosthetic graft, thereby rendering it unusable for dialysis. Although the thrombus can theoretically be removed, the underlying cause cannot. The patient thus enters a spiral phase of recurrent failure, hospitalization and surgery. Despite innumerable attempts of various kinds over the years to prevent this particular cause of graft thrombosis and secondary failure, there have been few substantive advances to date.

There is thus a widely recognized need for, and it would be highly advantageous to have a multiport vascular prosthesis, devoid of the above limitations.

### SUMMARY OF THE INVENTION

According to one aspect of the present invention there is provided a vascular prosthesis, comprising a primary tubular structure of non-woven polymer fibers and a secondary tubular structure of non-woven polymer fibers, the primary and the secondary tubular structures being in fluid communication via an anastomosis such that the primary tubular structure terminates at the anastomosis and the secondary tubular structure continues at the anastomosis.

According to another aspect of the present invention there is provided a method of forming an arteriovenous shunt in a vasculature of a subject, the method comprising: According to still further features in the described preferred embodiments the vascular prosthesis thereby creating a pair of vein ends; forming an opening in a wall of an artery of the vasculature; and connecting the secondary tubular structure to the pair of vein ends and the primary tubular structure to the opening in the wall of the artery, thereby forming the arteriovenous shunt.

According to further features in preferred embodiments of the invention described below, the anastomosis is characterized by an acute anastomosis angle. According to still further features in the described preferred embodiments the acute anastomosis angle is from about 10 degrees to about 70 degrees.

According to still further features in the described preferred embodiments the primary tubular structure has a profile selected such as to allow blood to enter the primary tubular structure from a primary input port thereof and flow through the secondary tubular structure in a predetermined direction.

According to still further features in the described preferred embodiments the diameter defining the primary tubular structure is larger at the anastomosis than far from the anastomosis.

According to still further features in the described preferred embodiments the diameter at the anastomosis is larger near the axial center of the primary tubular structure than far from the center.

According to still further features in the described preferred embodiments at least a part of the primary tubular structure is characterized by a cross section which is concave at one side of the anastomosis.

According to still further features in the described preferred embodiments at least a part of the primary tubular structure is characterized by a cross section which is concave at one side of the anastomosis and convex at an opposite side of the anastomosis.

According to still further features in the described preferred embodiments the vascular prosthesis is connected such that the concave side faces the downstream direction of the vein, and the convex side faces the upstream direction of the vein.

According to still further features in the described preferred embodiments the primary input port is outwardly concave.

According to still further features in the described preferred embodiments the secondary tubular structure comprises an output port being outwardly concave and located downstream the predetermined flow direction. According to still further features in the described preferred embodiments the secondary tubular structure comprises a secondary input port being outwardly concave and located upstream the predetermined flow direction.

According to still further features in the described preferred embodiments the vascular prosthesis further comprises a support structure extending from a primary input port of the primary tubular structure and the anastomosis. According to still further features in the described preferred embodiments the support structure further extends from a secondary input port of the secondary tubular structure and an output port of the secondary tubular structure.

According to still further features in the described preferred embodiments the support structure is embedded in the primary tubular structure and the secondary tubular structure.

According to still further features in the described preferred embodiments at least one of the primary and the secondary tubular structures comprises a plurality of layers each layer of the plurality of layers being made of non-woven polymer fibers.

According to still further features in the described preferred embodiments at least one layer includes a pharmaceutical agent incorporated therein for delivery of the pharmaceutical agent into the body vasculature during or after implantation of the vascular prosthesis within the body vasculature. According to still further features in the described preferred embodiments the pharmaceutical agent is included in any layer other than the outermost layer.

According to still further features in the described preferred embodiments at least one intermediate layer includes a thrombogenic agent incorporated therein, for delivery of the medicament into the body vasculature during or after implantation of the vascular prosthesis within the body vasculature.

According to an additional aspect of the present invention there is provided apparatus for manufacturing an electrospun multiport structure. The apparatus comprises, a substantially T-shaped precipitation electrode and a dispenser being at a first electric potential relative to the precipitation electrode and capable of dispensing a liquefied polymer to produce polymer fibers precipitating on the precipitation electrode, thereby to form the electrospun structure thereupon.

According to further features in preferred embodiments of the invention described below, the precipitation electrode comprises two detachable arms.

According to still further features in the described preferred embodiments the precipitation electrode comprises a removable connector for connecting the arms.

According to still further features in the described preferred embodiments the apparatus further comprising a subsidiary electrode system, being at a second potential relative to the precipitation electrode and configured to shape an electric field formed between the precipitation electrode and the dispenser.

According to still further features in the described preferred embodiments the apparatus further comprising a compartment, encapsulating the electrospinning system and the subsidiary electrode system, for keeping a clean environment within the compartment.

According to still further features in the described preferred embodiments the clean environment is characterized by class 1000 or cleaner.

According to still further features in the described preferred embodiments at least one of the dispenser and the precipitation electrode is operable to rotate such that a relative rotary motion is established between the dispenser and the precipitation electrode.

According to still further features in the described preferred embodiments at least one of the dispenser and the precipitation electrode is operable to move such that a relative linear motion is established between the dispenser and the precipitation electrode.

According to still further features in the described preferred embodiments the subsidiary electrode system comprises at least one planar electrode.

According to still further features in the described preferred embodiments the subsidiary electrode system comprises at least one cylindrical electrode.

According to still further features in the described preferred embodiments the subsidiary electrode system is repositionable with respect to the precipitation electrode.

The present invention successfully addresses the shortcomings of the presently known configurations by providing multiport vascular prosthesis and an apparatus for manufacturing multiport structures enjoying properties far exceeding the prior art.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawing. With specific reference now to the drawing in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawing making apparent to those skilled in the art how the several forms of the invention may be embodied in practice.

In the drawings:

FIG. 1 is a schematic illustration of a vascular prosthesis having a primary tubular structure and a secondary tubular structure, according to various exemplary embodiments of the present invention;

FIGS. 2a-b which are schematic planar views of the vascular prosthesis, according to various exemplary embodiments of the present invention;

FIG. 3 is a schematic illustration of the vascular prosthesis in a preferred embodiment in which the primary and/or secondary tubular structures include more than one layer of non-woven polymer fibers;

FIG. 4 a schematic illustration of the vascular prosthesis after implantation with the body vasculature, in a preferred embodiment in which the vascular prosthesis is used as an arteriovenous shunt;

FIG. 5 is a flowchart diagram of a method suitable for forming an arteriovenous shunt in a vasculature of a subject, according to various exemplary embodiments of the present invention.

FIGS. 6a-b are schematic illustration of an electrospinning apparatus (FIG. 6a) and a precipitation electrode (FIG. 6b), according to various exemplary embodiments of the present invention.

### DESCRIPTION OF THE PREFERRED EMBODIMENTS

The present embodiments comprise a device which can be used as a vascular prosthesis. Specifically, the present embodiments can be used to form an arteriovenous shunt in the vasculature of a subject.

The principles and operation of a vascular prosthesis according to the present embodiments may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

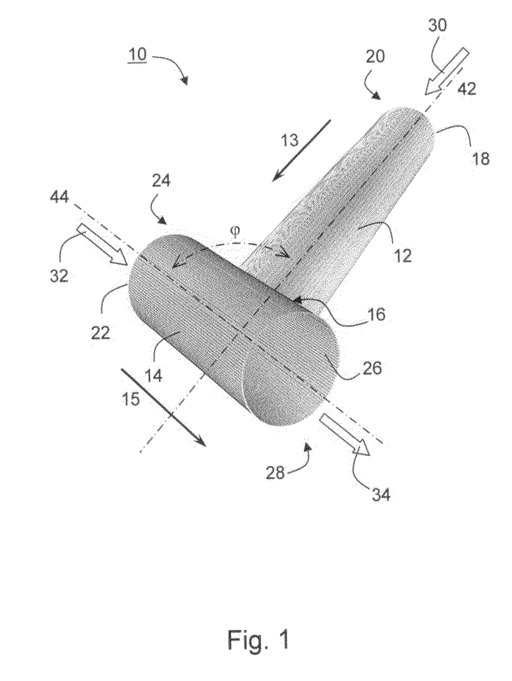

Referring now to the drawings, FIG. 1 illustrates a vascular prosthesis 10 according to various exemplary embodiments of the present invention. Vascular prosthesis 10 is a multiport vascular prosthesis which preferably comprises a primary tubular structure 12 having a longitudinal axis 42 and a secondary tubular structure 14 having a longitudinal axis 44. Each of structures 12 and 14 is formed of non-woven polymer fibers, which are preferably of nanometric thickness. Typical thickness of the polymer fibers is, without limitation, from about 50 nm to about 500 nm.

As used herein the term “about” refers to ±10%.

The polymer fibers can be manufactured using any technique for forming non-woven fibers, such as, but not limited to, an electrospinning technique, a wet spinning technique, a dry spinning technique, a gel spinning technique, a dispersion spinning technique, a reaction spinning technique or a tack spinning technique.

Suitable electrospinning techniques are disclosed, e.g., in International Patent Application, Publication Nos. WO 2002/049535, WO 2002/049536, WO 2002/049536, WO 2002/049678, WO 2002/074189, WO 2002/074190, WO 2002/074191, WO 2005/032400 and WO 2005/065578, the contents of which are hereby incorporated by reference.

Other spinning techniques are disclosed, e.g., U.S. Pat. Nos. 3,737,508, 3,950,478, 3,996,321, 4,189,336, 4,402,900, 4,421,707, 4,431,602, 4,557,732, 4,643,657, 4,804,511, 5,002,474, 5,122,329, 5,387,387, 5,667,743, 6,248,273 and 6,252,031 the contents of which are hereby incorporated by reference.

A preferred technique for manufacturing multiport vascular prosthesis suitable for the present embodiments is provided hereinunder.

Primary tubular structure 12 and secondary tubular structure 14 are in fluid communication via an anastomosis 16, such that primary structure 12 terminates at anastomosis 16 while secondary structure 14 continues at anastomosis 16. Thus, structure 12 has one free end (designated on FIG. 1 by numeral 20), and structure 14 has two free ends (designated on FIG. 1 by numeral 24 and 28). Anastomosis 16 is characterized by an anastomosis angle φ, which is conveniently defined as the acute angle between axes 42 and 44. Preferred values for φ are from about 10 degrees to about 70 degrees, more preferably from about 20 degrees to about 50 degrees.

Preferred internal diameter of the tubular structures is from about 1 mm to about 30 mm, more preferably from about 2 mm to about 20 mm, most preferably from about 2 mm to about 6 mm. Preferred wall thickness for said tubular structures is in the range between about 0.1 mm to about 2 mm, more preferably, between about 0.5 mm to about 1.5 mm.

Typically, but not obligatorily, the length of tubular structure 12 is larger than the length of tubular structure 14. A preferred length of tubular structure 12 is from about 1 cm to about 70 cm, more preferably from about 15 cm to about 40 cm. A preferred length of tubular structure 14 is from about 10 mm to about 40 mm, more preferably from about 15 mm to about 35 mm.

In use, prosthesis 10 preferably receives arterial blood flow 30 from an artery (not shown) and vein blood flow 32 from a vein (not shown). An output blood flow 34 which includes a mixture of blood 32 and blood 30 is supplied to the vein. As shown in FIG. 1, arterial blood flow 30 enters through a primary input port 18, located at the free end 20 of structure 12, and vein blood flow 32 enters through a secondary input port 22, located at free end 24 of structure 14. The blood flows through the lumens of structures 12 and 14 and exits through an output port 26, located at free end 28 of structure 14. According to a preferred embodiment of the present invention the acute side anastomosis 16 faces secondary input port 22 and the obtuse side anastomosis 16 faces output port 26. Vascular prosthesis 10 is therefore a multiport vascular prosthesis in the sense that it includes more than two ports (two input ports and one output port, in the present exemplary embodiment).

There is a preferred flow direction of blood through prosthesis 10. In primary structure 12 the preferred flow direction is into anastomosis 16, and in secondary structure 14 the preferred flow direction is into output port 26. The preferred flow directions are illustrated in FIG. 1 by arrows 13 (flow in structure 12) and 15 (flow in structure 12). As will be appreciated by one of ordinary skill in the art the desired flow direction is enabled by the natural blood pressure in the vasculature and the choice of anastomosis angle φ and. In particular, high arterial blood pressure prevents blood from flowing upstream within structure 12. In turn, and the acute anastomosis angle φ at the side of port 22 and the blood pressure from the veins (although being lower than the arterial pressure), directs the blood flow to output port 26.

While reducing the present invention to practice it has be uncovered that a proper blood flow through prosthesis 10 can be ensured by a judicious construction of the profile of the prosthesis. In particular it was found that the shape of the profile of primary structure 12 can facilitate blood flow from the primary structure 12 to secondary structure 14 (via anastomosis 16), such that the blood flows through structure 14 in direction 15.

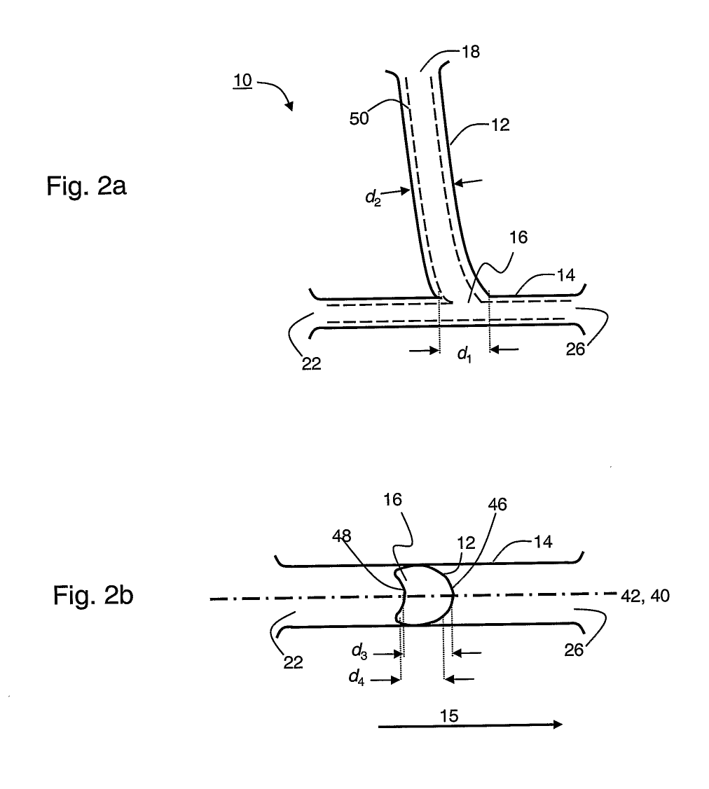

Reference is now made to FIGS. 2a-b, which are schematic planar views of prosthesis 10, where FIG. 2a is a top view of prosthesis 10, illustrating the profiles (longitudinal cross sections) of tubular structures 12 and 14, and FIG. 2b is a side view illustrating the transverse cross section of structure 12 and the profile of structure 14, according to various exemplary embodiments of the present invention. One skilled in the art will recognize that several numerals have been omitted from FIGS. 2a-b for clarity of presentation.

According to a preferred embodiment of the present invention primary structure 12 widens towards anastomosis 16. In other words, the diameter defining primary structure 12 is larger at or near anastomosis 16 than far from anastomosis 16. For example, referring to FIG. 2a, the diameter d₁ at or near anastomosis 16 is larger than the diameter d₂ about half the distance between anastomosis 16 and port 18. Additionally, referring to FIG. 2b, the diameter of structure 12 at anastomosis 16 is larger at a plane 40 defined by longitudinal axes 42 and 44 than far from plane 40. The diameter at plane 40 is designated in FIG. 2b by d₃ and the diameter farther from plane 40 is designated by d₄.

According to a preferred embodiment of the present invention at least a part of structure 12 (preferably the part near or at anastomosis 16) is characterized by a cross section which is concave at one side of anastomosis 16. More preferably, but not obligatorily, at least a part of the cross section of structure 12 is concave at one side of anastomosis 16 and convex at the opposite side thereof.

As used herein, “concave” and “convex” describe the contour of the inner wall of the tubular structure.

In the representative example of FIG. 2b, the cross section of structure 12 is concave at the side facing output port 26 and convex at the side facing secondary input port 22. The concave and convex parts of the cross section are designated in FIG. 2b by numerals 46 and 48, respectively.

In various exemplary embodiments of the invention each one of ports 18, 22 and 26 is independently concave outwardly, to facilitate entrance/exit of blood flow to prosthesis 10 and to reduce risk of blood coagulation near the ends of the tubular structures.

Optionally and preferably, prosthesis 10 can also comprise a support structure 50, extending from port 18 to anastomosis 16, and optionally also from port 22 to port 26. Support structure 50 can be disposed internally within the tubular structures, as illustrated in FIG. 2a, or it can be embedded in the walls of the tubular structures, as further detailed hereinbelow. Support structure 50 can be any support structure known in the art (to this end see, e.g., WO 02/49535 supra, and U.S. Pat. Nos. 6,945,993, 6,949,120 and 6,939,373). For example, structure 50 can be a deformable mesh of wires made of a metallic material such as, but not limited to, medical grade stainless steel or a material exhibiting temperature-activated shape memory properties, such as Nitinol.

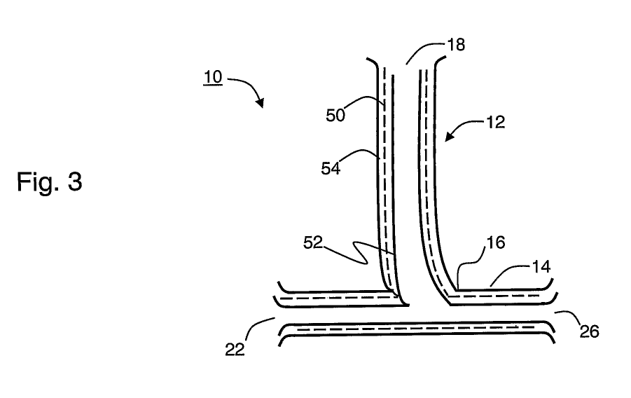

Reference is now made to FIG. 3, which is a schematic illustration of prosthesis 10 in a preferred embodiment in which the primary and/or secondary tubular structures include more than one layer of non-woven polymer fibers. Two layers, a liner layer 52 and a cover layer 54, are illustrated in FIG. 3, but it is not intended to limit the scope of the present invention to any particular number of layers. Specifically, one or both tubular structures can include three or more layers of non-woven polymer fibers. In the preferred embodiments in which support structure 50 is employed, it can be embedded between two successive layers.

The advantage of using a plurality of layers is that with such configuration each layer can have different properties, such as porosity and/or mechanical strength, depending on its function. For example, liner layer 52, which typically serves as a sealing layer to prevent bleeding, can be manufactured substantially as a smooth surface with relatively low porosity. Layer 52 thus prevents bleeding and preclotting. In addition, throughout the life of the vascular prosthesis, layer 52 ensures antithrombogenic properties and efficient endothelization of the inner surface of the vascular prosthesis. A typical thickness of layer 52 is from about 40 μm to about 200 μm, more preferably from about 60 μm to about 120 μm.

The requisite mechanical properties (high compliance, high breaking strength, etc.) of the vascular prosthesis are typically provided by the outer layers (e.g., cover layer 54). Thus, according to a preferred embodiment of the present invention the thickness of layer 54 is larger than the thickness of layer 52. A typical thickness of layer 52 is from about 50 μm to about 1000 μm.

Additionally, the porosity of layer 54 is preferably larger than the porosity of layer 52. A porous structure is known to promote ingrowth of surrounding tissues, which is extremely important for fast integration and long-term patency of the vascular prosthesis. When the vascular prosthesis comprises more than two layers, the porosity of the intermediate layers can differ from the porosities of the inner and outer layers. For example, the porosity of the layers can be a decreasing function of a distance of the layer from the center of the vascular prosthesis.

An additional advantage of the multilayer embodiment is that such configuration provides self-sealing properties to vascular prosthesis 10. This is particularly useful when prosthesis 10 is used as an arteriovenous shunt, in which case prosthesis 10 is punctured repeatedly. Self-sealing properties can be achieved by providing a vascular prosthesis with three or more layers, where the liner layer and the cover layer are formed from crude fibers with predominantly circumferential orientation, with relatively low porosity (say, from about 50% to about 70%), and the intermediate layer is formed from thin and randomly-oriented fibers, with relatively higher porosity (from about 80% to about 90%). Preferably, the intermediate layer comprises about 70% of the overall wall thickness of prosthesis 10. In accordance to the presently preferred embodiment of the invention, the inner layer and the outer layer, serve for supporting the intermediate layer.

Upon puncturing, a needle passes through the intermediate layer, by forcing the fibers apart, hence no rupturing occurs. The tearing is prevented due to the combination of high elasticity of the fibers, large number of voids and small number of bonds between the fibers. Once the needle extracted the original fibers web is reconstructed, both because of the fiber elasticity and because the pressure applied by the inner and outer layers. Thus, high level of sealing or re-annealing is achieved.

The strength properties of prosthesis 10 are mainly ensured by its inner and outer layers. The piercing damage in the outer and inner layers are spread apart of one another by a certain distance, thus minimizing the affect of puncture on the wall strength.

Drug delivery into the body vasculature can be performed during or after implantation of the vascular prosthesis within the body vasculature. Hence, according to a preferred embodiment of the present invention, one or more of the layers of prosthesis 10 incorporates a pharmaceutical agent for delivery of the pharmaceutical agent into the body vasculature during or after implantation of prosthesis 10. The pharmaceutical agent and its concentration can be selected in accordance with the expected pathology. For example, the implantation of the prosthesis may result in disorders such as restenosis, stenosis and hyper cell proliferation, in the blood vessel being in contact with the prosthesis. The incorporated pharmaceutical agent can therefore be a medicament for treating such and other disorders. In the preferred embodiments in which prosthesis 10 is used as an arteriovenous shunt for dialysis procedures, the incorporated pharmaceutical agent can be a thrombogenic agent to reduce or prevent hemorrhage following removal of the dialysis needle. Additionally or alternatively, the incorporated pharmaceutical agent can be an imaging agent to enable post implantation imaging.

With respect to hemorrhage prevention, it is recognized that factors which facilitate generation of haemostatic plug include adhesion and aggregation of platelets as well as formation of polymerized fibrin matrix at the site of vascular injury. The endothelial surface on the vessel wall is not thrombogenic. Vascular wall injury results in exposure of collagen and subendothelial proteins. The adherence of platelets to collagen is recognized as a critical initial event for generation of a haemostatic plug. The reason being the capturing of platelets from the flowing blood via rapid bond formation between their glycoprotein 1b receptor and von Willibrand factor immobilized on collagen.

In parallel with the platelets adhesion process, coagulation is initiated through release of tissue factor from the damaged vessel wall. Propagation of blood coagulation occurs by localized enzymatic complexes assembled on the plasma membrane of adherent platelets that expose negatively charged phospholipids. The thrombin thus formed further activates platelets and stabilizes the growing thrombus by the formation of fibrin.

Both platelets that are in direct contact with subendothelial collagen and platelets that form the main body of an adherent platelets thrombus can participate in the clot formation. The direct contact platelets are activated by collagen as well as by soluble agonists (such as thrombin). On the other hand, the platelets of the main body of the thrombus are activated by soluble agonists, with minimal or no collagen impact. According to a preferred embodiment of the present invention the thrombogenic agent is selected to affect the first phase of the thrombus formation so as to create weak clot formation and to occlude the holes in the artificial vessel.

Thrombogenic agents are preferably incorporated in one or more of the intermediate layers of prosthesis 10, other medicaments are preferably incorporated in one or more of the intermediate layers or the liner layer, and imaging agents can be incorporated in any of the layers.

Representative examples for suitable thrombogenic agents include, thrombin, a platelet activating factor or an analogue thereof, fibrin, factor V, factor IX, an antiphospholipid antibody or a portion thereof, copper or an alloy thereof, platinum or an alloy thereof a positively charged polymer (voltage being in the range between 0.2 and 0.8 volts), polyvinyl acrylate and cyanoacrylate.

Such agents are readily available in active or precursor form from a variety of suppliers. For example, thrombin or prothrombin can be obtained from Sigma-Aldrich.

According to presently preferred embodiments of the present invention, the thrombogenic agent is collagen (available from Sigma and BD Biosciences), von Willebrand Factor (preferably from a human source from HTI and American Diagnostica), thrombospondin (available from ProSpecTany TechnoGene and Sigma), tissue factor (available from Dade Behring and American Diagnostica), or various phospholipids (e.g. L-alpha Phosphatidylcholine, L-alpha-Phosphatidylserine, L-alpha-Phosphatidylethanolamine available from Avanti polar lipids)

To enable clotting of suture holes and more importantly of needle entry holes over a period of 12 months, the vascular graft of the present embodiments preferably includes a minimum recommended surface concentration of the thrombogenic agent per unit area of graft wall. Representative examples of preferred surface concentrations include, without limitation, from about 1 to about 20 pg/mm² foer for collagen, from about 1 to about 20 pg/mm² von Willebrand Factor, from about 1 to about 100 pg/mm² for Thrombospondin, from about 0.1 to about 5 pg/mm² for Tissue Factor.

Representative examples for suitable medicaments include, without limitation, heparin, tridodecylmethylammonium-heparin, epothilone A, epothilone B, rotomycine, ticlopidine, dexamethasone and caumadin.

Also contemplated are other pharmaceutical agents such as, but not limited to, antithrombotic, estrogens, corticosteroids, cytostatic, anticoagulant, vasodilator, antiplatelet, trombolytics, antimicrobials, antibiotics, antimitotics, antiproliferatives, antisecretory, nonsterodial antiflammentory, grow factor antagonists, free radical scavengers, antioxidants, radiopaque agents, immunosuppressive and radio-labeled agents.

Tubular structures 12 and 14 can be made of any known biocompatible polymer. In the layers which incorporate pharmaceutical agent, the polymer fibers are preferably a combination of a biodegradable polymer and a biostable polymer.

Suitable biostable polymers which can be used in the present embodiments include, without limitation, polycarbonate based aliphatic polyurethanes, silicon modificated polyurethanes, polydimethylsiloxane and other silicone rubbers, polyester, polyolefins, polymethyl-methacrylate, vinyl halide polymer and copolymers, polyvinyl aromatics, polyvinyl esters, polyamides, polyimides and polyethers.

Suitable biodegradable polymers which can be used in the present embodiments include, without limitation, poly (L-lactic acid), poly (lactide-co-glycolide), polycaprolactone, polyphosphate ester, poly (hydroxy-butyrate), poly (glycolic acid), poly (DL-lactic acid), poly (amino acid), cyanoacrylate, some copolymers and biomolecules such as collagen, DNA, silk, chitozan and cellulose.

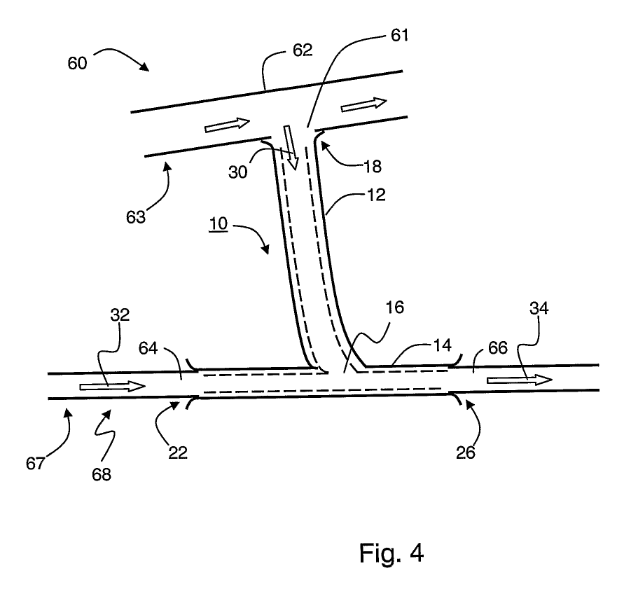

FIG. 4 is a schematic illustration of prosthesis 10 after implantation with the vasculature 60, in a preferred embodiment in which prosthesis 10 is used as an arteriovenous shunt. One skilled in the art will recognize that several numerals and components of prosthesis 10 have been omitted from FIG. 4 for clarity of presentation.

According to the presently preferred embodiment of the invention primary structure 12 is connected to an opening 61 formed in an artery 62, and secondary structure 14 is connected between an upstream side 64 and a downstream side 66 of a vein 68, such that secondary input port 22 is connected to upstream side 64 and output port 26 is connected to output port 24. In the embodiments in which the cross section of structure 12 is concave at one side of anastomosis 16 and convex at the opposite side thereof, prosthesis 10 is connected such that the concave side faces downstream 66, and the convex side faces upstream side 64 of vein 68. When blood is allowed to flow in artery 62 and vein 68, structure 12 receives arterial blood flow 30 from artery 62 and vein blood flow 32 from vein 68, and supplies output blood 34 to second side 66 of vein 68.

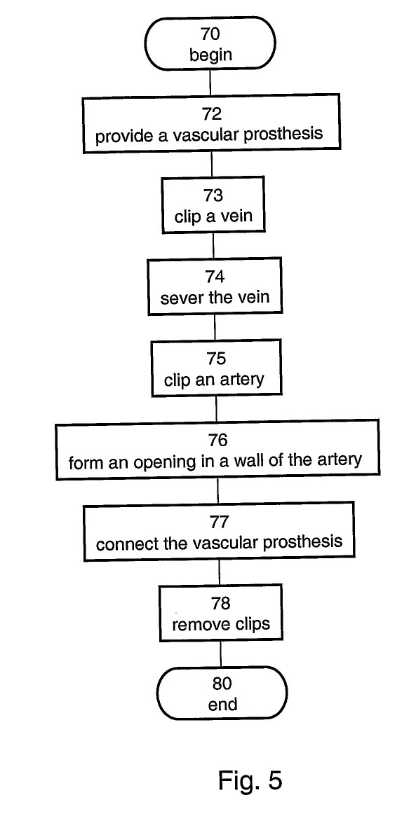

Reference is now made to FIG. 5, which is a flowchart diagram of a method suitable for forming an arteriovenous shunt in a vasculature of a subject. It is to be understood that, unless explicitly stated, the method steps described hereinbelow can be executed either contemporaneously or sequentially in many combinations or orders of execution. Specifically, the ordering of the flowchart of FIG. 5 is not to be considered as limiting. For example, two or more method steps, appearing in the following description or in the flowchart of FIG. 5 in a particular order, can be executed in a different order (e.g., a reverse order) or substantially contemporaneously.

The method begins at step 70 and continues to step 72 in which a vascular prosthesis (e.g., vascular prosthesis 10) is provided. The method continues to step 74 in which a vein is severed to create a pair of vein ends, one end at the upstream side of the vein and another end at the downstream end of the vein, see FIG. 4.

The method continues to step 76 in which an opening is formed in a wall of an artery of the vasculature. According to a preferred embodiment of the present invention each of steps 74 and 76 can be preceded by a step in which the respective blood vessel is clipped at an upstream location (see, for example, locations 63 and 67 in FIG. 4), so as to reduce blood lose during the procedure. The clipping of the vein and artery are represented by Blocks 73 and 75 of the flowchart diagram, respectively. the clipping. The method continues to step 77 in which the prosthesis is connected to the artery and the vein, as further detailed hereinabove and illustrated in FIG. 4.

If the clipping steps are employed, the method continues to step 78 in which the clips are removed to allow the blood to flow through the vascular prosthesis as further detailed hereinabove.

The method ends at step 80.

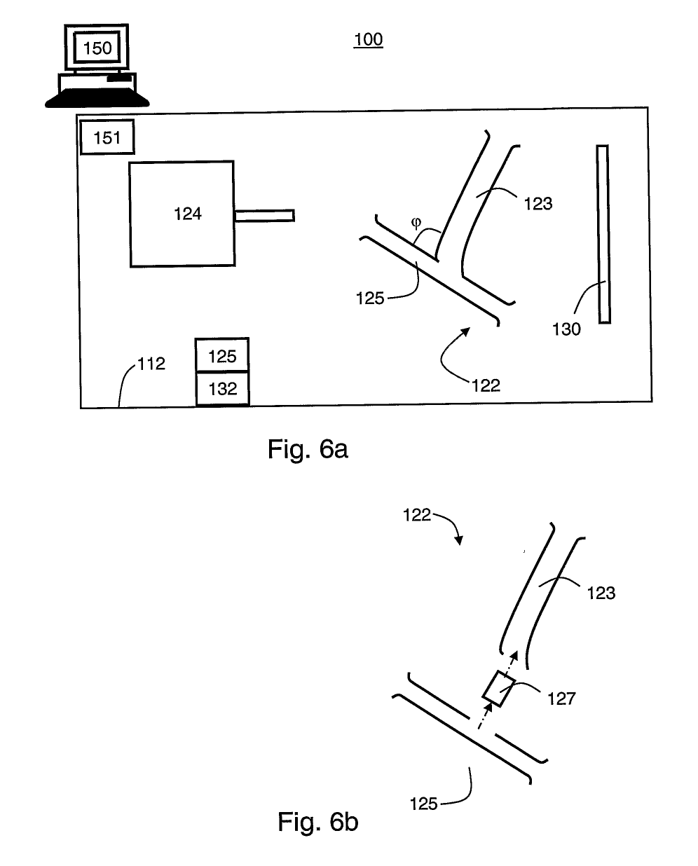

Reference is now made to FIGS. 6a-b, which are schematic illustrations of an apparatus 100 for manufacturing an multiport electrospun structure, according to various exemplary embodiments of the present invention. In its simplest configuration, apparatus 100 comprises a precipitation electrode 122, and a dispenser 124, positioned at a predetermined distance from precipitation electrode 122 and being kept at a first potential relative to precipitation electrode 122.

Precipitation electrode 122 is typically manufactured in accordance with the geometrical properties of the final product which is to be fabricated. In the representative example of FIG. 6, electrode 122 has a T-shape having arms 123 and 125 (arm 123 terminates on the side of arm 125), to enable manufacturing of multiport structures suitable, e.g., for implantation as arteriovenous shunts, as further detailed hereinabove. Electrode 122 can be made of, for example, stainless steel, or any other electrically conducting material.

The angle φ between arms 123 and 125 is not limited. Preferably, but not obligatorily, φ is below 70°. Electrode 122 is better illustrated in the explosion diagram of FIG. 6b. According to the presently preferred embodiment of the invention arms 123 and 125 of electrode 122 are detachable. For example, arms 123 and 125 can be connected by a removable end-to-side connector 127. The advantage of making arms 123 and 125 detachable is that such configuration facilitates the post manufacturing removal of the final electrospun product from electrode 122.

Alternatively, arms 123 and 125 can have a permanent connection therebetween, such that electrode 122 remains within the lumens of the final multiport structure. This embodiment is particularly when it is desired to manufacture a multiport structure having a mechanical support extending between its ports (such as, for example, support 50 hereinabove). Thus, electrode 122 can serve for post manufacture support of the multiport structure.

The potential difference between dispenser 124 and precipitation electrode 122 is preferably from about 10 kV to about 100 kV, typically about 60 kV. The potential difference between dispenser 124 and precipitation electrode 22 generate an electric field therebetween.

Dispenser 124 serves for dispensing a liquefied polymer in the electric field to produce polymer fibers precipitating on electrode 122. Precipitation electrode 122 serves for forming the electrospun structure thereupon.

In accordance with the electrospinning technique, a liquefied polymer is drawn into dispenser 124, and then, subjected to the electric field generated by the potential difference between dispenser 124 and electrode 122, is being charged and dispensed in a direction of electrode 122. Moving with high velocity in the inter-electrode space, jet of liquefied polymer cools or solvent therein evaporates, thus forming fibers which are collected on the surface of electrode 122.

According to a preferred embodiment of the present invention apparatus 100 comprises a subsidiary electrode system 130, which is preferably at a second potential relative to precipitation electrode 122 and configured to shape the aforementioned electric field. A typical potential difference between electrode 122 and electrode system 130 is from about 10 kV to about 100 kV, typically about 50 kV.

Subsidiary electrode system 130 controls the direction and magnitude of the electric field between precipitation electrode 122 and dispenser 124 and as such, can be used to control the orientation of polymer fibers precipitated on electrode 122. In some embodiments, subsidiary electrode system 130 serves as a supplementary screening electrode. Generally, the use of screening results in decreasing the coating precipitation factor, which is particularly important upon cylindrical precipitation electrodes having at least a section of small radii of curvature.

Electrode shapes which can be used in the present embodiments include, but are not limited to, a plane, a cylinder, a torus a rod, a knife, an arc or a ring.

Specifically, a cylindrical or planar subsidiary electrode enables manufacturing intricate-profile products being at least partially with small (from about 0.025 millimeters to about 5 millimeters) radius of curvature. Such subsidiary electrodes are also useful for achieving random or circumferential alignment of the fibers onto precipitation electrode 122.

Electrode system 130 may comprise a plurality of electrodes in any arrangement. The size, shape, position and number of electrodes in system 130 is preferably selected so as to maximize the coating precipitation factor, while minimizing the effect of corona discharge in the area of precipitation electrode 122 and/or so as to provide for controlled fiber orientation upon deposition.

In various exemplary embodiments of the invention system 130 comprises three cylindrical electrodes which can be of different diameters. For example, a large diameter cylindrical electrode can be positioned behind precipitation electrode 122 (with respect to dispenser 124), and two cylindrical electrodes of smaller diameter can be poisoned above and below electrode 122.

The ability to control fiber orientation is important when fabricating vascular prostheses in which a high radial strength and elasticity is important. It will be appreciated that a polar oriented structure can generally be obtained also by wet spinning methods, however in wet spinning methods the fibers are thicker than those used by electrospinning by at least an order of magnitude.

Control over fiber orientation is also advantageous when fabricating composite polymer fiber shells which are manufactured by sequential deposition of several different fiber materials.

Subsidiary electrodes of small radius of curvature, can be used to introduce distortion the electric field in an area adjacent to precipitation electrode 122. For maximal such effect, the diameter of the subsidiary electrode must be considerably smaller than that of precipitation electrode 122, yet large enough to avoid generation of a significant corona discharge.

According to a preferred embodiment of the present invention the position of any electrode of subsidiary electrode system 130 can be varied relative to precipitation electrode 122. Such design further facilitates the ability to control the electric field vector (intensity and direction) near electrode 122.

According to a preferred embodiment of the present invention apparatus 130 further comprises a compartment 112, dispenser 124, electrode 122 and subsidiary electrode system 130. Preferably, but not obligatorily, compartment 112 also encapsulates the power source 125 and circuitry 132 which supply the power to apparatus 100. Compartment 112 is preferably made of a material being transmissive in the visual range. Compartment 112 serves for keeping a clean environment therein. According to a preferred embodiment of the present invention the clean environment is of class 1000 (i.e., less than one thousands particles larger than 0.5 microns in each cubic foot of space) or cleaner. More preferably the clean environment is of class 100 (i.e., less than one thousand particles larger than 0.5 microns in each cubic foot of air space).

More preferably, compartment 112 serves as a climate chamber which besides the clean environment, maintains therein predetermined levels of other environmental conditions such as temperature and humidity.

Thus, according to a preferred embodiment of the present invention the temperature with compartment 112 is kept at a predetermined constant level within an accuracy of ±1° C., more preferably ±0.5° C. even more preferably ±0.2° C., so as to control and maintain the desired evaporation rate during the electrospinning process. Maintenance of accurate temperature within compartment 112 is advantageous because the thickness of the produced polymer fibers and the porosity of the electrospun structure, depends, inter alia, on the evaporation rate of solvent from the polymer jets emerge from dispenser 124. Preferred temperatures for the operation are from about 22° C. to about 40° C.

Additionally, the humidity within compartment 112 is maintained at a predetermined level to an accuracy of 5% more preferably 3% even more preferably 1%. Maintenance of accurate temperature within compartment 112 is useful for preventing or reducing formation of volume charge. Preferred humidity level, in relative value (the weight or pressure of moisture relative to the maximal weight or pressure of moisture for a given temperature) is about 40%.

Dispenser 124 and/or precipitation electrode 122 preferably rotate such that a relative rotary motion is established between dispenser 124 and electrode 122. Similarly, dispenser 124 and/or electrode 122 preferably move such that a relative linear motion is established between dispenser 124 and electrode 122. For example, in one preferred embodiment, precipitation electrode 122 rotates without performing a linear motion, while dispenser 124 performs a linear motion without performing a rotary motion. In another preferred embodiment, dispenser 124 rotates about electrode 122 and electrode 122 performs a linear reciprocal motion. In an additional preferred embodiment, dispenser 124 performs a spiral motion about electrode 122. The relative motion between dispenser 124 and electrode 122 can be established by any mechanism, such as, but not limited to, an electrical motor, an electromagnetic motor, a pneumatic motor, a hydraulic motor, a mechanical gear and the like.

In various exemplary embodiments of the invention apparatus 100 is controlled by a data processor 150 supplemented by an algorithm for controlling apparatus 100. Data processor 150 can communicate with any of the components of apparatus 100 directly or through a control unit 151 located within compartment 112. The communication can be via communication line or, more preferably, via wireless communication so as to preserve to clean environment in compartment 12. Preferably, but not obligatorily, processor 150 also communicates (e.g., through control unit 151) with source 125 and circuitry 132 for controlling the aforementioned potential differences and for automatically activating and deactivating apparatus 100. According to a preferred embodiment of the present invention processor 150 is configured (e.g., by a suitable computer program) to vary the relative rotary motion and/or relative linear motion between dispenser 124 and electrode 122. As will be appreciated by one ordinarily skilled in the art, different angular and/or linear relative velocities can result in different precipitation rates of polymer fibers on electrode 122. Thus, the computerized control on the motions can be used to select the desired precipitation rate, hence also the desired wall thickness of the electrospun structure.

Additionally, processor 150 can signal the mechanism for establishing the linear and/or angular motions of dispenser 124 and/or electrode 122 to change the corresponding velocities, at a given instant or instances of the process. This embodiment is particularly useful when manufacturing multilayer structures. Thus, by selecting different motion characteristics of dispenser 124 and/or electrode 122 for different layers, the electrospinning process for each layer is at a different precipitation rate, resulting in a different density of fibers on the formed layer. Since the porosity of the layer depends on the density of fiber, such process can be used for manufacturing multilayer electrospun structures in which the layers have predetermined and different porosities. Additionally, each layer can have a different wall thickness, which can also be controlled as further detailed above.

For example, when the electrospun structure is a vascular prosthesis, it can be made of two or more layer, whereby the inner layer can be substantially smooth (with small porosity) and the outer layer has a larger porosity, as further detailed hereinabove.

In various exemplary embodiments of the invention, a multiport electrospun structure, e.g., such as vascular prosthesis 10, is manufactured as follows.

One or more liquefied polymers are provided and introduced into the dispenser. The liquefied polymer(s) can also be mixed with one or more conductivity control agents or charge control agents for improving the interaction of the fibers with the electric field. The distance between the precipitation electrode and the subsidiary electrodes, the distance between the dispenser and the precipitation electrode, and the angle between the dispenser and the precipitation electrode are adjusted by the adjustments mechanism and recorded into the data processor.

The dispenser, precipitation electrode and subsidiary electrode system are sealed by the compartment and the appropriate environmental conditions are established.

Parameters, such as, but not limited to, wall thickness, number of layer, angular and linear velocities, temperature, hydrostatic pressure, polymer viscosities, and the like, are recorded into the data processor which Also recorded are the types of polymers.

Apparatus 100 is activated and the liquefied polymer is extruded under the action of the hydrostatic pressure through the spinnerets. As soon as meniscus of the extruded liquefied polymer forms, a process of solvent evaporation or cooling starts, which is accompanied by the creation of capsules with a semi-rigid envelope or crust. Because the liquefied polymer possesses a certain degree of electrical conductivity, the capsules become charged by the electric field. Electric forces of repulsion within the capsules lead to a drastic increase in hydrostatic pressure. The semi-rigid envelopes are stretched, and a number of point micro-ruptures are formed on the surface of each envelope leading to spraying of ultra-thin jets of the liquefied polymer from the spinnerets.

Under the effect of a Coulomb force, the jets depart from the dispenser and travel towards the opposite polarity electrode, i.e., the precipitation electrode. Moving with high velocity in the inter-electrode space, the jet cools or solvent therein evaporates, thus forming fibers which are collected on the surface of the precipitation electrode.

Once a first layer is formed, the data processor signals the dispenser to reselect a different liquefied polymer (in embodiments in which different liquefied polymers are used for different layers), and the motion mechanisms to change the rotary and/or linear velocities (in embodiments in which different the layers have different wall thicknesses and/or different porosities). The signaling of the data processor is preferably performed without ceasing the electrospinning process, such that the new layer is formed substantially immediately after the previous layer.

Once all the layers are formed, the compartment is opened and the precipitation electrode, including the electrospun structure formed thereupon is disengaged from the system. The electrospun multiport structure is then removed from the precipitation electrode.

According to a preferred embodiment of the present invention the removal of the multiport structure is performed as follows. The precipitation electrode, including the electrospun structure is irradiated by ultrasound radiation. It was found by the inventor of the present invention that ultrasound radiation facilitates the removal of the electrospun structure from the electrode. Additionally and more preferably, the precipitation electrode including the electrospun structure can also be subjected to at least one substantially abrupt temperature change. The abrupt temperature change can be applied by any suitable heat carrier, including, without limitation, a liquid bath. The removal process can also be controlled by the data processor. Specifically, the data processor can control the duration and level of the applied temperatures and/or the ultrasound radiation.

The precipitation electrode including the multiport structure is immersed in an ultrasonic bath of low temperature (about 0° C.) for a first predetermined period (about 1-10 minutes, more preferably 3-5 minutes). Subsequently, the precipitation electrode including the multiport structure is immersed in another ultrasonic bath of high temperature (from about 40° C. to about 100° C.) for a second predetermined period (about 1-10 minutes, more preferably 3-5 minutes). According to a preferred embodiment of the present invention once the above thermal treatment is completed the arms of the precipitation electrode are detached (preferably while the multiport structure is on the precipitation electrode). In an alternative embodiment, the detachment of the arms can precede the thermal treatment. Irrespectively, each arm of the precipitation electrode is separately pulled out from the multiport structure.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A vascular prosthesis, comprising a primary tubular structure of non-woven polymer fibers and a secondary tubular structure of non-woven polymer fibers, said primary and said secondary tubular structures being in fluid communication via an anastomosis such that said primary tubular structure terminates at said anastomosis and said secondary tubular structure continues at said anastomosis.

2. A method of forming an arteriovenous shunt in a vasculature of a subject, the method comprising:
providing the vascular prosthesis of claim 1;
severing a vein of said vasculature, thereby creating a pair of vein ends;
forming an opening in a wall of an artery of said vasculature; and
connecting said secondary tubular structure to said pair of vein ends and said primary tubular structure to said opening in said wall of said artery, thereby forming the arteriovenous shunt.

3. The vascular prosthesis or method of claim 1, wherein said anastomosis is characterized by an acute anastomosis angle.

4. The vascular prosthesis or method of claim 1, wherein said primary tubular structure has a profile selected such as to allow blood to enter said primary tubular structure from a primary input port thereof and flow through said secondary tubular structure in a predetermined direction.

5. The vascular prosthesis or method of claim 4, wherein the diameter defining said primary tubular structure is larger at said anastomosis than far from said anastomosis.

6. The vascular prosthesis or method of claim 5, wherein said diameter at said anastomosis is larger near the axial center of said primary tubular structure than far from said center.

7. The vascular prosthesis or method of claim 5, wherein at least a part of said primary tubular structure is characterized by a cross section which is concave at one side of said anastomosis.

8. The vascular prosthesis or method of claim 5, wherein at least a part of said primary tubular structure is characterized by a cross section which is concave at one side of said anastomosis and convex at an opposite side of said anastomosis.

9. The method of claim 8, wherein the vascular prosthesis is connected such that said concave side faces the downstream direction of said vein, and said convex side faces the upstream direction of said vein.

10. The vascular prosthesis or method of claim 4, wherein said primary input port is outwardly concave.

11. The vascular prosthesis or method of claim 4, wherein said secondary tubular structure comprises an output port being outwardly concave and located downstream said predetermined direction.

12. The vascular prosthesis or method of claim 4, wherein said secondary tubular structure comprises a secondary input port being outwardly concave and located upstream said predetermined direction.

13. The vascular prosthesis or method of claim 1, wherein the vascular prosthesis further comprises a support structure extending from a primary input port of said primary tubular structure and said anastomosis.

14. The vascular prosthesis or method of claim 13, wherein said support structure further extends from a secondary input port of said secondary tubular structure and an output port of said secondary tubular structure.

15. The vascular prosthesis or method of claim 13, wherein said support structure is embedded in said primary tubular structure and said secondary tubular structure.

16. The vascular prosthesis or method of claim 1, wherein at least one of said primary and said secondary tubular structures comprises a plurality of layers each layer of said plurality of layers being made of non-woven polymer fibers.

17. The vascular prosthesis or method of claim 16, wherein at least one layer of said plurality of layers includes a pharmaceutical agent incorporated therein for delivery of said pharmaceutical agent into the body vasculature during or after implantation of the vascular prosthesis within said body vasculature.

18. The vascular prosthesis or method of claim 17, wherein said at least one layer is other than the outermost layer of said plurality of layers.

19. The vascular prosthesis or method of claim 16, wherein at least one intermediate layer of said plurality of layers includes a thrombogenic agent incorporated therein, for delivery of said medicament into the body vasculature during or after implantation of the vascular prosthesis within said body vasculature.

20. The vascular prosthesis or method of claim 3, wherein said acute anastomosis angle is from about 10 degrees to about 70 degrees.

21. Apparatus for manufacturing an electrospun multiport structure, comprising, a substantially T-shaped precipitation electrode and a dispenser being at a first electric potential relative to said precipitation electrode and capable of dispensing a liquefied polymer to produce polymer fibers precipitating on said precipitation electrode, thereby to form the electrospun structure thereupon.

22. The apparatus of claim 21, wherein said precipitation electrode comprises two detachable arms.

23. The apparatus of claim 22, wherein said precipitation electrode comprises a removable connector for connecting said arms.

24. The apparatus of claim 21, further comprising a subsidiary electrode system, being at a second potential relative to said precipitation electrode and configured to shape an electric field formed between said precipitation electrode and said dispenser.

25. The apparatus of claim 21, further comprising a compartment, encapsulating said electrospinning system and said subsidiary electrode system, for keeping a clean environment within said compartment.

26. The apparatus of claim 25, wherein said clean environment is characterized by class 1000 or cleaner.

27. The apparatus of claim 21, wherein at least one of said dispenser and said precipitation electrode is operable to rotate such that a relative rotary motion is established between said dispenser and said precipitation electrode.

28. The apparatus of claim 21, wherein at least one of said dispenser and said precipitation electrode is operable to move such that a relative linear motion is established between said dispenser and said precipitation electrode.

29. The apparatus of claim 24, wherein said subsidiary electrode system comprises at least one planar electrode.

30. The apparatus of claim 24, wherein said subsidiary electrode system comprises at least one cylindrical electrode.

31. The apparatus of claim 24, wherein said subsidiary electrode system is repositionable with respect to said precipitation electrode.
